(19) United States
(12) Patent Application Publication
(10) Pub. No.: US 2015/0320705 A1
(43) Pub. Date: Nov. 12, 2015

(54) METHOD OF INHIBITING OR TREATING AMYOTROPHIC LATERAL SCLEROSIS WITH PHENOXYALKYLCARBOXYLIC ACIDS

(71) Applicant: MediciNova, Inc., La Jolla, CA (US)

(72) Inventors: Kazuko MATSUDA, Beverly Hills, CA (US); Yuichi IWAKI, La Jolla, CA (US)

(73) Assignee: MediciNova, Inc., La Jolla, CA (US)

(21) Appl. No.: 14/705,610

(22) Filed: May 6, 2015

Related U.S. Application Data
(60) Provisional application No. 61/990,583, filed on May 8, 2014.
(60) Provisional application No. 62/029,260, filed on Jul. 25, 2014.

(51) Int. Cl. A61K 31/192

(52) U.S. Cl. CPC A61K 31/192

(57) ABSTRACT

A compound of Formula (I), or a metabolite thereof, or an ester of the compound of Formula (I) or the metabolite thereof, or a pharmaceutically acceptable salt of each thereof

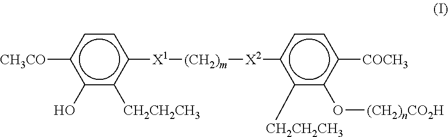

wherein: m is an integer from 2 to 5 inclusive; and n is an integer from 3 to 8 inclusive; and X¹ and X² are each independently sulfur, oxygen, a sulfinyl group or a sulfonyl group, provided that X¹ and X² are not simultaneously oxygen. Such a compound may be useful for treating, inhibiting, or preventing the progression of amyotrophic lateral sclerosis, primary lateral sclerosis, or familial amyotrophic lateral sclerosis, or a symptom of each thereof.

### REFERENCE TO RELATED APPLICATIONS

This application claims priority to U.S. provisional application No. 61/990,583 filed May 8, 2014, and 62/029,260 filed Jul. 25, 2014, the content of each of which is incorporated herein by reference.

### FIELD

This technology relates to treating, inhibiting, or preventing the progression of amyotrophic lateral sclerosis, primary lateral sclerosis, or familial amyotrophic lateral sclerosis, or a symptom of each thereof, conditions leading to or arising from them, and/or negative effects of each thereof by administering phenoxyalkylcarboxylic acids.

### BACKGROUND

Amyotrophic lateral sclerosis (ALS) also referred to as Lou Gehrig's disease, is a rapidly progressive, fatal neurological disease that attacks the neurons responsible for controlling voluntary muscles. Subjects with ALS frequently die from respiratory failure, usually within 3 to 5 years from the onset of symptoms. As many as 20,000-30,000 people in the U.S. alone have ALS, and an estimated 5,000 people in the U.S. are diagnosed with the disease each year. ALS is one of the most common neuromuscular diseases worldwide, and people of all races and ethnic backgrounds are affected. ALS most commonly affects people between 40 and 60 years of age, but younger and older people also can develop the disease. Men are affected more often than women.

### SUMMARY

In one aspect, a method is provided for treating, inhibiting, or preventing the progression of a disorder selected from amyotrophic lateral sclerosis (ALS), primary lateral sclerosis (PLS), and familial ALS, or a symptom thereof in a patient suffering therefrom, the method comprising administering to the patient an effective amount of a compound of Formula (I):

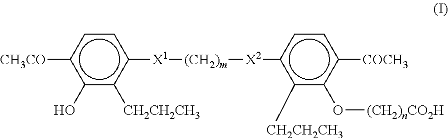

or a metabolite thereof, or an ester of the compound of Formula (I) or the metabolite thereof, or a pharmaceutically acceptable salt of each thereof, wherein m is an integer from 2 to 5 inclusive, and n is an integer from 3 to 8 inclusive, X¹ and X² each independently represent sulfur, oxygen, a sulfinyl group or a sulfonyl group, provided that X¹ and X² are not simultaneously oxygen.

In one embodiment, the disorder treated is ALS or a symptom thereof. In another embodiment, the disorder treated is PLS or a symptom thereof. In another embodiment, the disorder treated is familial ALS or a symptom thereof.

In another embodiment, the disorder inhibited is ALS or a symptom thereof. In another embodiment, the disorder inhibited is PLS or a symptom thereof. In another embodiment, the disorder inhibited is familial ALS or a symptom thereof.

In another embodiment, the disorder whose progression is prevented is ALS or a symptom thereof. In another embodiment, the disorder whose progression is prevented is PLS or a symptom thereof. In another embodiment, the disorder whose progression is prevented is familial ALS or a symptom thereof.

In one embodiment, the compound of Formula (I) is a compound of Formula (IA) (or MN-001):

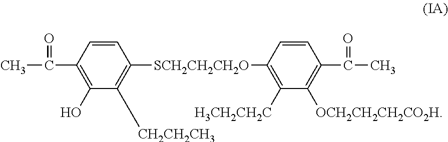

In another embodiment, the metabolite of the compound of Formula (I) and (IA) is a compound of Formula (IB) (or MN-002):

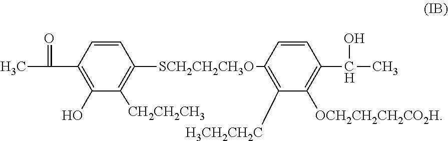

### BRIEF DESCRIPTION OF THE FIGURES

FIG. 1 graphically illustrates comparative life span in Sod1 mutants (Sod1⁻) and wt (Sod1+). Maximum life span of mutants is 25-30 days compared to 70-80 days for controls; recovering on life span curve can indicate positive compound activity.

FIG. 2 graphically illustrates high (% viability) sensitivity of SOD1-null adults compared to a wild stock after exposition of adult flies to 2 mmol of paraquat. Resistance to paraquat treatment can indicate positive activity of the compound tested.

FIG. 3 graphically illustrates that flies with interfered SOD gene (DMSO), show lower survival percentages after paraquat exposure (See SOD-DMSO and SOD-No Paraquat) and that treatment with either of the two positive control compounds (the anti-SMA compound riluzole or the antioxidant vitamin E) increased this percentage, as did. MN-001 in a dose dependent manner.

FIG. 4 graphically illustrates percent viability at 29° C. of at least 150 flies of each genotype analyzed: F1 of wild-type cross, F1 of VAPB mutant cross; and VAPB mutant in stock. ***p-value<0.0001 were calculated with a t-student test using the Graph pad program.

### DETAILED DESCRIPTION

As used herein, and in the appended claims, the singular forms “a,” “an” and “the” include plural references unless the context clearly dictates otherwise.

“Administering” or “administration of” a drug to a patient (and grammatical equivalents of this phrase) includes both direct administration, including self-administration, and indirect administration, including the act of prescribing a drug. For example, as used herein, a physician who instructs a patient to self-administer a drug and/or provides a patient with a prescription for a drug is administering the drug to the patient.

“C_{X}” when placed before a group refers to the number of carbon atoms in that group to be X.

“Alkyl” refers to a monovalent acyclic hydrocarbyl radical having 1 to 12 carbon atoms. Non-limiting examples of alkyl include methyl, ethyl, propyl, isopropyl, butyl, isobutyl, tertiary butyl, pentyl, hexyl and the like.

“Aryl” refers to a monovalent aromatic hydrocarbyl radical having up to 10 carbon atoms. Non-limiting examples of aryl include phenyl and naphthyl.

“Heteroaryl” refers to an aromatic group of from 1 to 10 carbon atoms and 1 to 4 heteroatoms selected from the group consisting of oxygen, nitrogen, sulfur within the aromatic ring, wherein the nitrogen and/or sulfur atom(s) of the heteroaryl are optionally oxidized (e.g., N-oxide, —S(O)— or —S(O)₂—). Such heteroaryl groups can have a single ring (e.g., pyridyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. Non limiting examples of heteroaryl include pyridyl, pyrrolyl, indolyl, thiophenyl, and furyl.

“Cycloalkyl” refers to a monovalent non-aromatic cyclic hydrocarbyl radical having 3-12 carbon atoms. Non-limiting examples of cycloalkyl include cyclopropyl, cyclobutyl, cyclopentyl, cyclohexyl, and the like.

“Heterocyclyl” refers to a monovalent non-aromatic cyclic group of 1 to 10 carbon atoms and 1 to 4 heteroatoms selected from the group consisting of oxygen, nitrogen, sulfur within the cycle, wherein the nitrogen and/or sulfur atom(s) of the heteroaryl are optionally oxidized (e.g., N-oxide, —S(O)— or —S(O)₂—). Such heteroaryl groups can have a single ring (e.g., piperidinyl or tetrahydrofuranyl) or multiple condensed rings wherein the condensed rings may or may not be aromatic and/or contain a heteroatom provided that the point of attachment is through an atom of the non-aromatic heterocyclyl group. Non limiting examples of heterocyclyl include pyrrolidinyl, piperidinyl, piperazinyl, and the like.

“Amino” refers to —NH₂.

“Alkylamino” refers to —NHR_{B}, wherein R_{B} is C₁-C₆ alkyl optionally substituted with 1-3 aryl, heteroaryl, cycloalkyl, or heterocyclyl group.

“Dialkylamino” refers to —N(R_{B})₂, wherein R_{B} is defined as above.

“Comprising” shall mean that the methods and compositions include the recited elements, but not exclude others. “Consisting essentially of” when used to define methods and compositions, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transitional terms and phrases are within the scope of this invention.

“Effective amount” of a compound utilized herein is an amount that, when administered to a patient treated as herein, will have the intended therapeutic effect, e.g., alleviation, amelioration, palliation or elimination of one or more manifestations of the medical condition in the patient. The full therapeutic effect does not necessarily occur by administration of one dose (or dosage), and may occur only after administration of a series of doses. Thus, an effective amount may be administered in one or more administrations.

“Amyotrophic lateral sclerosis (ALS)” also referred to as Lou Gehrig's disease, is a rapidly progressive, fatal neurological disease that attacks the nerve neurons responsible for controlling voluntary muscles (muscle action we are able to control, such as those in the arms, legs, and face). The disease belongs to a group of disorders known as motor neuron diseases, which are characterized by the gradual degeneration and death of motor neurons.

ALS causes weakness with a wide range of disabilities. Eventually, all muscles under voluntary control are affected, and individuals lose their strength and the ability to move their arms, legs, and body. When muscles in the diaphragm and chest wall fail, people lose the ability to breathe without ventilatory support. Most people with ALS die from respiratory failure, usually within 3 to 5 years from the onset of symptoms.

The earliest symptoms may include fasciculations, cramps, tight and stiff muscles (spasticity), muscle weakness affecting an arm or a leg, slurred and nasal speech, or difficulty chewing or swallowing. These general complaints then develop into more apparent weakness or atrophy.

Muscle weakness and atrophy spread to other parts of the body as the disease progresses. Individuals may develop problems with moving, swallowing (dysphagia), and speaking or forming words (dysarthria). Symptoms of upper motor neuron involvement include spasticity and exaggerated reflexes (hyperreflexia) including an overactive gag reflex. An abnormal reflex commonly called Babinski's sign (the large toe extends upward as the sole of the foot is stimulated in a certain way) also indicates upper motor neuron damage. Symptoms of lower motor neuron degeneration include muscle weakness and atrophy, muscle cramps, and fasciculations. To be diagnosed with ALS, people have signs and symptoms of both upper and lower motor neuron damage that is not attributed to other causes.

“Familial ALS” accounts for approximately 5 to 10 percent of all ALS cases, with the rest being sporadic (idiopathic) in origin. The presence of atypical features such as young age of onset, sensory loss, and a positive family history of ALS, other neurodegenerative disorders, and dementia indicates a possibility of familial ALS.

“Primary lateral sclerosis or (PLS)” is a neuromuscular disease with slowly progressive weakness in voluntary muscle movement. PLS is a motor neuron disease. PLS affects the upper motor neurons (also called corticospinal neurons) in the arms, legs, and face. PLS often affects the legs first, followed by the body, trunk, arms and hands, and, finally the bulbar muscles (muscles that control speech, swallowing, and chewing). Symptoms include weakness, muscle stiffness and spasticity, clumsiness, slowing of movement, and problems with balance and speech. PLS is more common in men than in women, with a varied gradual onset that generally occurs between ages 40 and 60. PLS progresses gradually over a number of years, or even decades. PLS is not considered to have a hereditary cause.

“Pharmaceutically acceptable” refers to non-toxic and suitable for administration to a patient, including a human patient.

“Pharmaceutically acceptable salts” refer to salts that are non-toxic and are suitable for administration to patients. Non-limiting examples include alkali metal, alkaline earth metal, and various primary, secondary, and tertiary ammonium salts. When the ester of the compound of Formula (I) includes a cationic portion, for example, when the ester includes an amino acid ester, the salts thereof can include various carboxylic acid, sulfonic acid, and miner acid salts. Certain non-limiting examples of salts include sodium, potassium, and calcium salts.

“Protecting groups” refer to well-known functional groups which, when bound to a functional group, render the resulting protected functional group inert to the reaction to be conducted on other portions of a compound and the corresponding reaction condition, and which can be reacted to regenerate the original functionality under de-protection conditions. The protecting group is selected to be compatible with the remainder of the molecule. A “carboxylic acid protecting group” protects the carboxylic functionality of the phenoxyalkylcarboxylic acids during their synthesis. Non limiting examples of carboxylic acid protecting groups include benzyl, p-methoxybenzyl, p-nitrobenzyl, allyl, benzhydryl, and trityl. Additional examples of carboxylic acid protecting groups are found in standard reference works such as Greene and Wuts, Protective Groups in Organic Synthesis., 2d Ed., 1991, John Wiley & Sons, and McOmie Protective Groups in Organic Chemistry, 1975, Plenum Press. Methods for protecting and de-protecting the carboxylic acids disclosed herein can be found in the art, and specifically in Greene and Wuts, supra, and the references cited therein.

“Treating” a medical condition or a patient refers to taking steps to obtain beneficial or desired results, including clinical results. For purposes of the various aspects and embodiments of the present invention, beneficial or desired clinical results include, but are not limited to, reduction, alleviation, or amelioration of one or more manifestations of or negative effects of ALS, PLS or familial ALS, improvement in one or more clinical outcomes, diminishment of extent of sclerosis, delay or slowing of sclerosis progression, amelioration, palliation, or stabilization of the scleroses state, and other beneficial results described herein.

Provided herein are methods administering an effective amount of a compound of Formula (I):

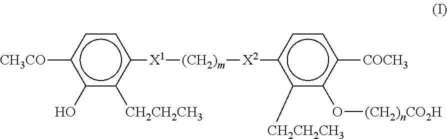

or a metabolite thereof, or an ester of the compound of Formula (I) or the metabolite thereof, or a pharmaceutically acceptable salt of each thereof, wherein the variables are defined as herein.

As used herein, “a metabolite thereof” refers to a metabolite that shows substantially similar therapeutic activity as a compound of Formula (I). Non-limiting examples of such metabolites include compounds where the —COCH₃ group, of a compound of Formula (I), that is attached to the phenyl containing the —O—(CH₂)ₙCO₂H moiety is metabolized to a 1-hydroxyethyl (—CH(OH)Me) group.

Metabolites containing such a 1-hydroxyethyl group contain an asymmetric center on the 1-position of the 1-hydroxyethyl group. The corresponding enantiomers and mixtures thereof, including racemic mixtures, are included within the metabolites of the compound of Formula (I) as utilized herein.

As used herein, “an ester thereof” refers to an ester of the phenolic hydroxy group and/or an ester of the carboxylic acid shown in the compound of Formula (I), and an ester of the 1-hydroxyethyl (an aliphatic hydroxy group) group of a metabolite of the compound Formula (I). An ester of the phenolic and/or the aliphatic hydroxy groups can include, without limitation, as the corresponding acid, a carboxylic acid R_{A}—CO₂H, wherein R_{A} is C₁-C₆ alkyl, aryl, heteroaryl, C₃-C₁₂ cycloalkyl, or C₂-C₈ heterocyclyl, wherein the alkyl, aryl, heteroaryl, cycloalkyl, or heterocyclyl are optionally substituted with from 1 to 4 C₁-C₃ alkyl, aryl, CO₂H, amino, alkylamino, or dialkylamino groups. Other acids such as mono-, di-, or tri phosphoric acids are also contemplated. An ester of the carboxylic acid can include, without limitation, as the corresponding alcohol, a compound of formula R_{A}—OH, wherein R_{A} is defined as above. In one embodiment, only the carboxylic acid in Formula (I) is esterified. In another embodiment, only the phenolic hydroxy group in Formula (I) is esterified. In another embodiment, R_{A} is C₁-C₄ alkyl. As will be apparent to the skilled artisan, such esters act as prodrugs that are hydrolyzed in vivo to release the compound of Formula (I) or a salt thereof.

In another embodiment, the compound of Formula (I) is a compound of Formula (IA):

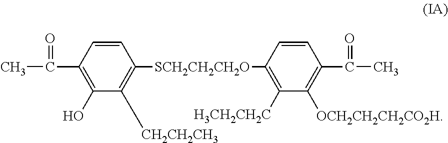

In another embodiment, the metabolite of the compound of Formula (I) and (IA) is a compound of Formula (IB):

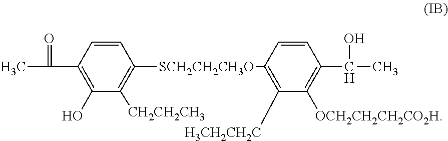

The compound may be administered orally. For example, the compound may be administered as a tablet or a capsule. In another embodiment, the compound of Formula (IA) is present in polymorphic form A that is substantially free of other polymorphic forms. In another embodiment, the compound is administered as a liquid dosage form. In another embodiment, the compound is administered in an amount from about 100 to about 4,000 mg/day, divided into one, two, or three portions.

The efficacy of a compound or composition utilized herein can be demonstrated by methods well-known to the skilled artisan. For example, the methods provided can be tested in animal models of amyotrophic lateral sclerosis (ALS) well known to the skilled artisan. Mouse models such as motor neuron degeneration (Mnd), progressive motor neuronopathy (pmn), wobbler, and a canine model, such as, hereditary canine spinal muscular atrophy (HCSMA) can be employed for these purposes. Drosophila fruit fly or transgenic mouse overexpressing the mutated SOD1 gene of familial ALS patients, can be used to demonstrate the usefulness of the methods provided herein. A study of selected features from various models can demonstrate further usefulness of the methods provided herein.

The synthesis and certain biological activity of the compounds of Formula (I) are described in U.S. Pat. No. 4,985,585 which is incorporated herein in its entirety by reference. For example, the compound of Formula (IA) is prepared by reacting a phenol of Formula (II):

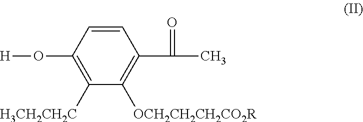

wherein, R is a carboxylic acid protecting group, with a compound of Formula (III):

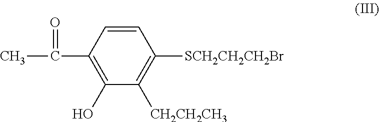

to provide a compound of Formula (IC):

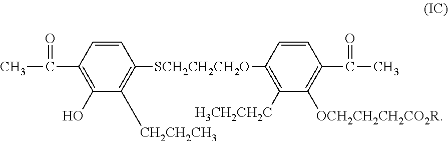

Non-limiting examples of acid protecting groups, or R groups, include C₁-C₆ alkyl, benzyl, benzhydryl, and trityl, wherein the benzyl, benzhydryl, or trityl group is optionally substituted with from 1 to 6 C₁-C₆ alkyl, halo, and/or C₁-C₆ alkoxy groups. The leaving group, other than the bromo group of Formula (III), may be used. Non-limiting examples of such other leaving groups include, but are not limited to, chloro and tosylate.

De-protection of the protected carboxylic acid of Formula (IC) provides the compound of Formula (IA). Compounds of Formula (IC) may be useful in accordance with any of the described methods and compounds. Non-limiting examples of de-protection methods include, but are not limited to, alkaline hydrolysis and hydrogenolysis under H₂ and a catalyst such as Pd/C or Pt/C.

The reactions may be carried out in an inert organic solvent. Such solvents include, but are not limited to, methylethylketone, diethylketone, or dimethylformamide. The nucleophilic displacement reaction may be conducted at a temperature below room temperature up to the reflux temperature of the solvent, in the presence of an inorganic base, such as potassium carbonate or sodium carbonate, and optionally in the presence of potassium iodide. The reactions are carried out for a period of time sufficient to provide substantial product as determined by well-known methods such as thin layer chromatography and ¹H-NMR. Other compounds utilized herein are made by following the procedures described herein and upon appropriate substitution of starting materials, and/or following methods well known to the skilled artisan. See also, U.S. Pat. No. 5,290,812 (incorporated herein in its entirety by reference).

The compound of Formula (IA) is recrystallized under controlled conditions to provide an essentially pure orthorhombic polymorph, referred to as Form A crystals (e.g., 90% or more, preferably at least 95% Form A). Polymorphic Form A and processes for producing it are described in U.S. Pat. Nos. 7,060,854 and 7,064,146; which are incorporated herein in their entirety by reference. All polymorphic forms of the compound of Formula (I) are active, but polymorphic Form A is preferred. Under certain conditions, the solubility and the bioavailability of this polymorph are superior to the other polymorphs and thus Form A may offer improved solid formulations.

Form A crystals can be obtained, for example, by dissolving the compound of Formula (IA) in 5 to 10 parts by weight of ethanol at 25° C. to 40° C., to give a yellow to orange solution. The ethanol solution is charged with 1 to 10 parts of water and agitated at 20° C. to 25° C. for about 15 to 60 minutes and then at 5° C. to 10° C. for an additional period of from 1 to 4 hours, preferably 2.0 to 3.0 hours, resulting in an off-white suspension. To this suspension is added 5 to 15 parts of water and the mixture is agitated at 5° C. to 10° C. for an additional from 1 to 4 hours, preferably 1.5 to 2.0 hours. A solid, white to off-white product is isolated by vacuum filtration and the filter cake is washed with water and dried in a vacuum at 25° C. to 40° C. for 12 to 24 hours.

For compounds utilized herein that exist in enantiomeric forms, such as certain metabolites of the compound of Formula (I) (for example, the compound of formula IB), the two enantiomers can be optically resolved. Such a resolution may be performed, for example, and without limitation, by forming diastereomeric salt of a base such as (S)-(−)-1-(1-naphthyl) ethylamine with the corresponding carboxylic acid compound, or by separating the enantiomers using chiral column chromatography. Intermediates to such compounds, which intermediates also exist in enantiomeric forms can be similarly resolved.

Any of the compounds may be administered orally; or intravenously, intramuscularly, or subcutaneously by injection; or transdermally. Effective dosage levels can vary widely from about 100 to about 4000 mg per day. In one embodiment, the daily dosage range is 250 to 2,000 mg, given in one, two, or three portions. In one embodiment, the daily dosage range is 100 to 500 mg, such as 100, 200, 300, 400, or 500 mg given in one, two, or three portions. In one embodiment, the daily dosage range is 250 to 2,000 mg, such as 250, 500, 750, 1,000, 1,250, 1,500, 1,750, or 2,000 mg given in one, two, or three portions. In one embodiment, the daily dosage range is 1,000 to 4,000 mg, such as 1,000, 2,000, 3,000, or 4,000 mg, given in one, two, or three portions. In another embodiment, the dosage is 1000 mg twice a day. In other embodiments, suitable dosages include 1,000 mg qd, 1,000 mg bid, and 750 mg tid.

Actual amounts will depend on the circumstances of the patient being treated. As those skilled in the art recognize, many factors that modify the action of the active substance will be taken into account by the treating physician such as the age, body weight, sex, diet and condition of the patient, the time of administration, the rate and route of administration. Optimal dosages for a given set of conditions can be ascertained by those skilled in the art using conventional dosage determination tests.

The compounds utilized herein can be formulated in any pharmaceutically acceptable form, including liquids, powders, creams, emulsions, pills, troches, suppositories, suspensions, solutions, and the like. Therapeutic compositions containing the compounds utilized herein will ordinarily be formulated with one or more pharmaceutically acceptable ingredients in accordance with known and established practice. In general, tablets are formed utilizing a carrier such as modified starch, alone or in combination with carboxymethyl cellulose (Avicel), for example at about 10% by weight. The formulations are compressed at from 1,000 to 3,000 pounds pressure in the tablet forming process. The tablets preferably exhibit an average hardness of about 1.5 to 8.0 kp/cm², preferably 5.0 to 7.5 kp/cm². Disintegration time varies from about 30 seconds to about 15 or 20 minutes.

Formulations for oral use can be provided as hard gelatin capsules wherein the therapeutically active compounds utilized herein are mixed with an inert solid diluent such as calcium carbonate, calcium phosphate or kaolin, or as soft gelatin capsules in which the compounds are mixed with an oleaginous medium, e.g., liquid paraffin or olive oil. Suitable carriers include magnesium carbonate, magnesium stearate, talc, sugar, lactose, pectin, dextrin, starch, gelatin, tragacanth, methylcellulose, sodium carboxymethyl cellulose, a low melting wax, cocoa butter, and the like.

The compounds utilized herein can be formulated as aqueous suspensions in admixture with pharmaceutically acceptable excipients such as suspending agents including, but not limited to, sodium carboxymethyl cellulose, methylcellulose, hydroxypropylmethyl cellulose, sodium alginate, polyvinylpyrrolidone, gum tragacanth and gum acacia; dispersing or wetting agents such as naturally occurring phosphatide, e.g., lecithin, or condensation products of an alkaline oxide with fatty acids, e.g., polyoxyethylene stearate, or condensation products of ethylene oxide with long chain aliphatic alcohols, e.g, heptadecaethylene-oxycetanol, or condensation products of ethylene oxide with partial esters derived from fatty acids and a hexitol, e.g., polyoxyethylene sorbitol monoleate or condensation products of ethylene oxide with partial esters derived from fatty acids and hexitol anhydrides, e.g., polyoxyethylene sorbitan monoleate. Such aqueous suspensions can also contain one or more preservatives, e.g., ethyl- or n-propyl-p-hydroxy benzoate, one or more coloring agents, one or more flavoring agents and one or more sweetening agents, such as glycerol, sorbitol, sucrose, saccharin or sodium or calcium cyclamate.

Suitable formulations also include sustained release dosage forms, such as those described in U.S. Pat. Nos. 4,788,055; 4,816,264; 4,828,836; 4,834,965; 4,834,985; 4,996,047; 5,071,646; and, 5,133,974, the contents of which are incorporated herein in their entirety by reference.

Other forms suitable for oral administration include liquid form preparations including emulsions, syrups, elixirs, aqueous solutions, or solid form preparations which are intended to be converted shortly before use to liquid form preparations. Emulsions may be prepared in solutions, for example, in aqueous propylene glycol solutions or may contain emulsifying agents, for example, such as lecithin, sorbitan monooleate, or acacia. Aqueous solutions can be prepared by dissolving the active component in water and adding suitable colorants, flavors, stabilizing, and thickening agents. Solid form preparations may contain, in addition to the active component, colorants, flavors, stabilizers, buffers, artificial and natural sweeteners, dispersants, thickeners, solubilizing agents, and the like.

The compounds utilized herein may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example as solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

The compounds utilized herein may be formulated for nasal administration. The solutions or suspensions are applied directly to the nasal cavity by conventional means, for example, with a dropper, pipette or spray. The formulations may be provided in a single or multidose form. The patient can administer an appropriate, predetermined volume of the solution or suspension via a dropper or pipette. A spray may be administered for example by means of a metering atomizing spray pump.

The compounds utilized herein may be formulated for aerosol administration, particularly to the respiratory tract and including intranasal administration. The compound will generally have a small particle size for example of the order of 5 microns or less. Such a particle size may be obtained by means known in the art, for example by micronization. The active ingredient is provided in a pressurized pack with a suitable propellant such as a chlorofluorocarbon (CFC), (for example, dichlorodifluoromethane, trichlorofluoromethane, or dichlorotetrafluoroethane), carbon dioxide or other suitable gases. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine. The powder carrier will form a gel in the nasal cavity. The powder composition may be presented in unit dose form for example in capsules or cartridges of, for example gelatin or blister packs from which the powder may be administered by means of an inhaler.

The compounds utilized herein may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges including active agents in a flavored base, usually sucrose and acacia or tragacanth; pastilles including the active ingredient in an inert base such as gelatin and glycerin or sucrose and acacia; and mouthwashes including the active ingredient in a suitable liquid carrier.

The compounds utilized herein may be formulated for administration as suppositories. In such a formulation, a low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds utilized herein may be formulated for vaginal administration. Pessaries, tampons, creams, gels, pastes, foams or sprays containing in addition to the active ingredient such carriers as are known in the art to be appropriate.

When desired, formulations can be prepared with enteric coatings adapted for sustained or controlled release administration of the active ingredient. A common type of controlled release formulation that may be used for the purposes of the present invention comprises an inert core, such as a sugar sphere, a first layer, coated with an inner drug-containing second layer, and an outer membrane or third layer controlling drug release from the inner layer.

The cores are preferably of a water-soluble or swellable material, and may be any such material that is conventionally used as cores or any other pharmaceutically acceptable water-soluble or water-swellable material made into beads or pellets. The cores may be spheres of materials such as sucrose/starch (Sugar Spheres NF), sucrose crystals, or extruded and dried spheres typically comprised of excipients such as microcrystalline cellulose and lactose.

The substantially water-insoluble material in the first layer is generally a “GI insoluble” or “GI partially insoluble” film forming polymer (dispersed or dissolved in a solvent). As examples may be mentioned ethyl cellulose, cellulose acetate, cellulose acetate butyrate, polymethacrylates such as ethyl acrylate/methyl methacrylate copolymer (Eudragit NE-30-D) and ammonio methacrylate copolymer types A and B (Eudragit RL3OD and RS30D), and silicone elastomers. Usually, a plasticizer is used together with the polymer. Exemplary plasticizers include: dibutylsebacate, propylene glycol, triethylcitrate, tributylcitrate, castor oil, acetylated monoglycerides, acetyl triethylcitrate, acetyl butylcitrate, diethyl phthalate, dibutyl phthalate, triacetin, fractionated coconut oil (medium-chain triglycerides).

The second layer containing the active ingredient may be comprised of the active ingredient (drug) with or without a polymer as a binder. The binder, when used, is usually hydrophilic but may be water-soluble or water-insoluble. Exemplary polymers to be used in the second layer containing the active drug are hydrophilic polymers such as polyvinylpyrrolidone, polyalkylene glycol such as polyethylene glycol, gelatine, polyvinyl alcohol, starch and derivatives thereof, cellulose derivatives, such as hydroxypropylmethyl cellulose (HPMC), hydroxypropyl cellulose, carboxymethyl cellulose, methyl cellulose, ethyl cellulose, hydroxyethyl cellulose, carboxyethyl cellulose, carboxymethyl hydroxyethyl cellulose, acrylic acid polymers, polymethacrylates, or any other pharmaceutically acceptable polymer. The ratio of drug to hydrophilic polymer in the second layer is usually in the range of from 1:100 to 100:1 (w/w).

Suitable polymers for use in the third layer, or membrane, for controlling the drug release may be selected from water insoluble polymers or polymers with pH-dependent solubility, such as, for example, ethyl cellulose, hydroxypropylmethyl cellulose phthalate, cellulose acetate phthalate, cellulose acetate trimellitate, polymethacrylates, or mixtures thereof, optionally combined with plasticizers, such as those mentioned above.

Optionally, the controlled release layer comprises, in addition to the polymers above, another substance(s) with different solubility characteristics, to adjust the permeability, and thereby the release rate, of the controlled release layer. Exemplary polymers that may be used as a modifier together with, for example, ethyl cellulose include: HPMC, hydroxyethyl cellulose, hydroxypropyl cellulose, methylcellulose, carboxymethylcellulose, polyethylene glycol, polyvinylpyrrolidone (PVP), polyvinyl alcohol, polymers with pH-dependent solubility, such as cellulose acetate phthalate or ammonio methacrylate copolymer and methacrylic acid copolymer, or mixtures thereof. Additives such as sucrose, lactose and pharmaceutical grade surfactants may also be included in the controlled release layer, if desired.

Also provided herein are unit dosage forms of the formulations. In such forms, the formulation is subdivided into unit dosages containing appropriate quantities of the active component (e.g., and without limitation, a compound of Formula (I) or an ester thereof, or a salt of each thereof). The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Other suitable pharmaceutical carriers and their formulations are described in Remington: The Science and Practice of Pharmacy 1995, edited by E. W. Martin, Mack Publishing Company, 19th edition, Easton, Pa.

The present invention, thus generally described, will be understood more readily by reference to the following examples, which are provided by way of illustration and are not intended to be limiting of the present invention.

### EXAMPLES

### Example 1

### Drosophila Life Span Assay as an ALS Treatment Model

Drosophila males will be collected. Flies will be transferred to fresh food (with compound) every 2-3 days. The number of living flies is analyzed daily. The experiment is performed under temperature controlled conditions (25° C.) and uses negative controls (only solvent), and positive controls (wt stock, any antioxidant compound reported as able to increase life span in this fly model). In order to compare the activity of the testing compound with riluzole (an FDA-approved drug for ALS), this drug will be added to the assay.

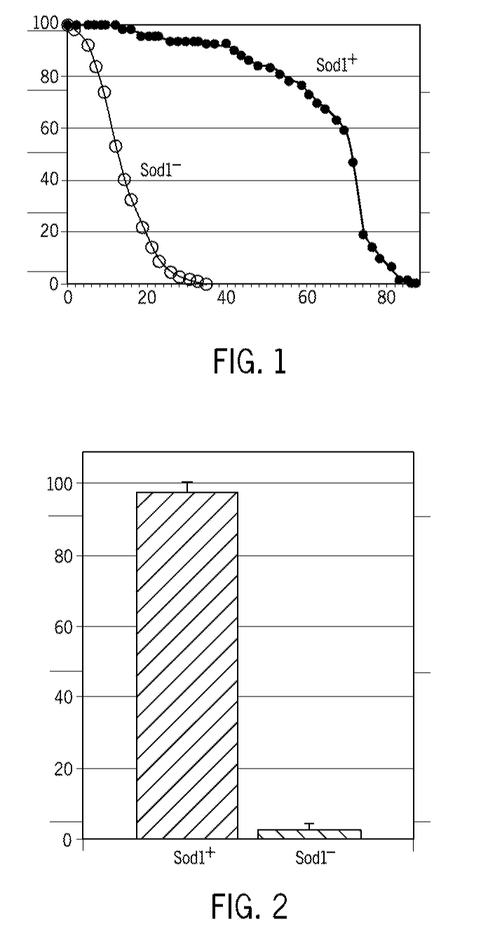

The experiment includes the analysis of four compound concentrations (10, 100, 250 and 1000 μM) and will evaluate 240 flies for each concentration (16 replicates with 15 flies each one). Recovering on life span curve can indicate positive compound activity. See FIG. 1.

Compound requirement: 10-15 mg of MN-001/MN-002 will be tested.
Timing: 5 months (1-2 months to expand the fly stock and 3 months for assay execution and results interpretation).

### Example 2

### Drosophila Paraquat Sensitivity Assay as an ALS Treatment Model

Drosophila males will be collected and keep on fly food for 24 h. Then flies will be transferred to vials containing 3-mm paper filter disks saturated with 250 μl of 1% sucrose containing 2 mM paraquat or 1% sucrose, 2 mM paraquat and the tested compound. The vials will be stored at 25° C. in the dark, and flies are enumerated after 24 h.

Three replicas for each concentration will be performed in the same day and three replicas of the assay will be performed in different days. A negative control (only solvent), and positive controls (wt stock, any antioxidant compound reported as able to increase life span in this fly model), and riluzole will be added to the assay.

The experiment includes the analysis of four compound concentrations (10, 100, 250 and 1000 μM) and will evaluate 360 flies for each concentration (8 replicates×3 days with 15 flies each one). Resistance to paraquat treatment will be indicative of positive activity of the compound tested. See, FIG. 2.

Compound requirement: 1-3 mg of MN-001/MN-002 will be tested.
Timing: 10 weeks (1-2 months to expand the fly stock, two weeks for assay execution and results interpretation)

### Results:

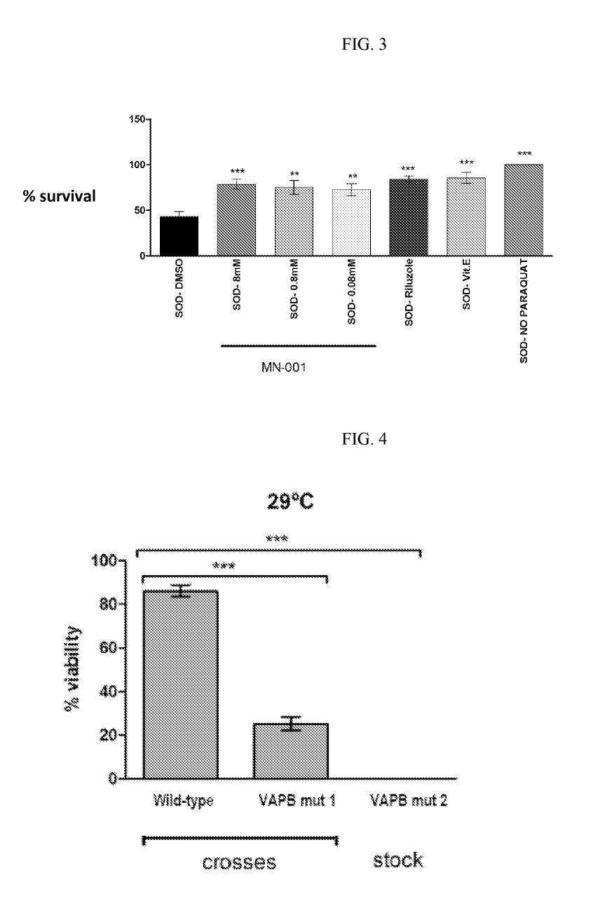

MN-001 was tested at concentrations of 0.08 mM, 0.8 mM, 8 mM (DMSO). As tested, MN-001 reduced paraquat toxicity on SOD deficient flies in a dose dependent manner (see FIG. 3). Flies with interfered SOD gene (DMSO), show lower survival (or survivorship) percentages after paraquat exposure (See SOD-DMSO and SOD-No Paraquat). Treatment with either the two positive compounds (the anti-SMA compound riluzole or the antioxidant vitamin E) increased this percentage. MN-001 also increased this survival in a dose dependent manner.

### Example 3

### Evaluation of Anti-ALS Activity on VAP-33a Drosophila Mutants

From other mutant stocks available and involving other ALS linked genes, loss of function of Vap-33-1 gene (excision of transcribed sequence and loss of protein function) displays valid fly phenotypes for evaluation of compounds activity. Indistinctly, Vap-33A^{Δ448} or Vap-33A^{Δ20} stocks display neurophysiology defects linked to a lethal phenotype during larvae development.

### Viability Assay

Vap-33A^{Δ} mutants are larval lethal with rare adult escapers (˜1%). Embryos or larvae at stage 1 will be seeded on fly food with different compound concentrations (10, 25, 100 μM). Three replicas for each concentration will be performed in the same day. Three replicas of the assay will be performed in different days. Number of adult escapers will be quantified after 14 days of compound treatment. A negative control (only solvent), and positive controls (wt stock, any antioxidant compound reported as able to increase life span in this fly model), and riluzole will be added to the assay.

The experiment includes the analysis of four compound concentrations (10, 100, 250 and 1000 μM) and will evaluate 180 flies for each concentration (4 replicates×3 days with 15 flies each one).

Compound requirement: 5-10 mg of MN-001/MN-002 will be tested.
Timing: 3 months (2 months to expand the fly stock, 1 month for assay execution and results interpretation

### Example 4

### Evaluation of Anti-ALS Activity on VAPB Drosophila Mutants

A fly-based ALS model based on VAPB gene was employed. These mutant flies displayed a reduced viability as a significant phenotype, as graphically illustrated in FIG. 3. FIG. 3 shows the percent viability at 29° C. of at least 150 flies of each genotype analyzed: F1 of wild-type cross, F1 of VAPB mutant cross; and VAPB mutant in stock. At least five tubes seeded with fifteen L1 larvae each were analyzed each day. Data was collected during two independent days. ***p-value<0.0001 calculated with a t-student test using the Graph pad program.

MN-001 was tested in the model along with DMSO, riluzole, and vitamin D. Three replicates were tested for riluzole and vitamin D (45 larvae seeded), and eight for compound B and DMSO (120 larvae seeded).

The following results were obtained under the test conditions. No adult flies were observed from DMSO and riluzole. From vitamin D, 1 adult was observed in one of the 3 replicates. For MN-001, 5 of 8 wells displayed adults but only 1 adult in each of them, thereby demonstrating MN-001's ability to increase viability in a statistically significant manner.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

What is claimed is:

1. A method of treating a disorder selected from amyotrophic lateral sclerosis (ALS), primary lateral sclerosis (PLS), and familial ALS, or a symptom thereof in a patient suffering therefrom, the method comprising administering to the patient an effective amount of a compound of Formula (I), or metabolite thereof, or an ester thereof, or a metabolite of the ester thereof, or a pharmaceutically acceptable salt of each thereof: 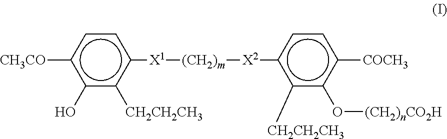
wherein: m is an integer from 2 to 5 inclusive; and n is an integer from 3 to 8 inclusive; and X¹ and X² are each independently sulfur, oxygen, a sulfinyl group or a sulfonyl group, provided that X¹ and X² are not simultaneously oxygen.

2. The method of claim 1, wherein the compound of Formula (I) is of Formula (IA)

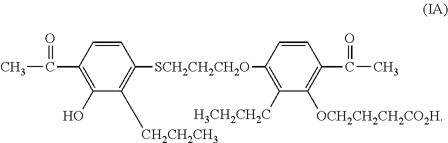

3. The method of claim 1, wherein the metabolite of the compound of Formula (I) is a compound of Formula (IB):

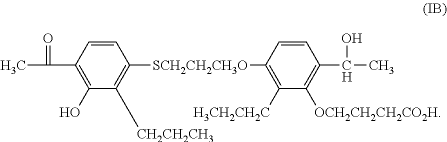

4. The method of claim 1, wherein the compound is administered orally.

5. The method of claim 4, wherein the compound is administered as a tablet or a capsule.

6. The method of claim 2, wherein the compound is present in an orthorhombic polymorphic form A that is substantially free of other polymorphic forms.

7. The method of claim 1, wherein the compound is administered as a liquid dosage form.

8. The method of claim 1, wherein the compound is administered in an amount from about 100 to about 4,000 mg/day, divided into one, two, or three portions.

9. The method of claim 1, wherein the disorder treated is ALS or a symptom thereof.

10. The method of claim 1, wherein the disorder treated is PLS or a symptom thereof.

11. The method of claim 1, wherein the disorder treated is familial ALS or a symptom thereof.

12. A method of inhibiting a disorder selected from ALS, PLS, and familial ALS, or a symptom thereof in a patient suffering therefrom, the method comprising: administering to the patient an effective amount of a compound of Formula (I), or a metabolite thereof, or an ester of the compound of Formula (I) or the metabolite thereof, or a pharmaceutically acceptable salt of each thereof: 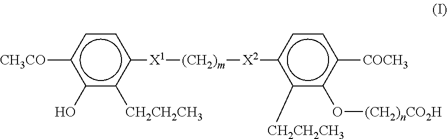
wherein: m is an integer from 2 to 5 inclusive; n is an integer from 3 to 8 inclusive; and X¹ and X² are each independently sulfur, oxygen, a sulfinyl group or a sulfonyl group, provided that X¹ and X² are not simultaneously oxygen.

13. The method of claim 12, wherein the compound of Formula (I) is of Formula (IA)

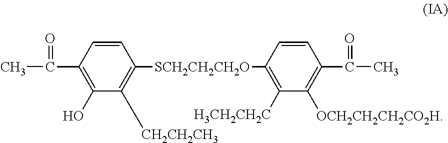

14. The method of claim 12, wherein the metabolite of the compound of Formula (I) is a compound of Formula (IB):

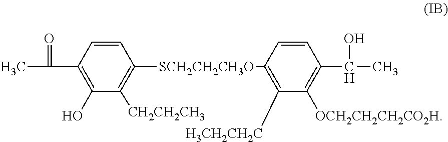

15. The method of claim 12, wherein the compound is administered orally.

16. The method of claim 15, wherein the compound is administered as a tablet or a capsule.

17. The method of claim 13, wherein the compound is present in an orthorhombic polymorphic form A that is substantially free of other polymorphic forms.

18. The method of claim 12, wherein the compound is administered as a liquid dosage form.

19. The method of claim 12, wherein the compound is administered in an amount from about 100 to about 4,000 mg/day, divided into one, two, or three portions.

20. The method of claim 12, wherein the disorder inhibited is ALS or a symptom thereof.

21. The method of claim 12, wherein the disorder inhibited is PLS or a symptom thereof.

22. The method of claim 12, wherein the disorder inhibited is familial ALS or a symptom thereof.

23. A method of preventing the progression of a disorder selected from ALS, PLS, and familial ALS, or a symptom thereof in a patient suffering therefrom, the method comprising administering to the patient an effective amount of a compound of Formula (I), or a metabolite thereof, or an ester of the compound of Formula (I) or the metabolite thereof, or a pharmaceutically acceptable salt of each thereof: 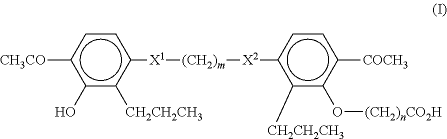
wherein: m is an integer from 2 to 5 inclusive; and n is an integer from 3 to 8 inclusive; and X¹ and X² are each independently sulfur, oxygen, a sulfinyl group or a sulfonyl group, provided that X¹ and X² are not simultaneously oxygen.

24. The method of claim 23, wherein the compound of Formula (I) is of Formula (IA)

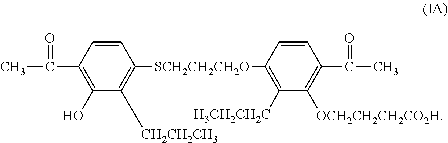

25. The method of claim 23, wherein the metabolite of the compound of Formula (I) is a compound of Formula (IB):

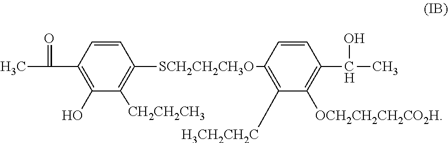

26. The method of claim 23, wherein the compound is administered orally.

27. The method of claim 26, wherein the compound is administered as a tablet or a capsule.

28. The method of claim 24, wherein the compound is present in an orthorhombic polymorphic form A that is substantially free of other polymorphic forms.

29. The method of claim 23, wherein the compound is administered as a liquid dosage form.

30. The method of claim 23, wherein the compound is administered in an amount from about 100 to about 4,000 mg/day, divided into one, two, or three portions.

31. The method of claim 23, wherein the disorder for which progression is prevented is ALS or a symptom thereof.

32. The method of claim 23, wherein the disorder for which progression is prevented is PLS or a symptom thereof.

33. The method of claim 23, wherein the disorder for which progression is prevented is familial ALS or a symptom thereof.
